SEQUENCE LISTING (7 sequences)

SEQ ID NO: 1 - DNA, 40 bp, synthetic construct
  source 1..40: mol_type = other DNA
acgacgtttgcgatgagaaacgtatggtttcgaaggtcgt

SEQ ID NO: 2 - DNA, 47 bp, synthetic construct
  source 1..47: mol_type = other DNA
ctctcgacgacgtttgcgatgagaaacgtatggtttcgaaggtcgtc

SEQ ID NO: 3 - DNA, 12 bp, synthetic construct
  source 1..12: mol_type = other DNA
agtcgtcgagag

SEQ ID NO: 4 - protein, 406 aa, Thermotoga maritima
MIIGNCLILKDFSSEPFWGAVEIENGTIKRVLQGEVKVDLDLSGKLVMPALFNTHTHAPMTLLRGVAEDLSFEEWLFSKVLPIEDRLTEKMAYYGTILAQMEMARHGIAGFVDMYFHEEWIAKAVRDFGMRALLTRGLVDSNGDDGGRLEENLKLYNEWNGFEGRIFVGFGPHSPYLCSEEYLKRVFDTAKSLNAPVTIHLYETSKEEYDLEDILNIGLKEVKTIAAHCVHLPERYFGVLKDIPFFVSHNPASNLKLGNGIAPVQRMIEHGMKVTLGTDGAASNNSLNLFFEMRLASLLQKAQNPRNLDVNTCLKMVTYDGAQAMGFKSGKIEEGWNADLVVIDLDLPEMFPVQNIKNHLVHAFSGEVFATMVAGKWIYFDGEYPTIDSEEVKRELARIEKELYSS

SEQ ID NO: 5 - protein, 230 aa, Batis maritima
MSTVANIAPVFTGDCKTIPTPEECATFLYKVVNSGGWEKCWVEEVIPWDLGVPTPLVLHLVKNNALPNGKGLVPGCGGGYDVVAMANPERFMVGLDISENALKKARETFSTMPNSSCFSFVKEDVFTWRPEQPFDFIFDYVFFCAIDPKMRPAWGKAMYELLKPDGELITLMYPITNHEGGPPFSVSESEYEKVLVPLGFKQLSLEDYSDLAVEPRKGKEKLARWKKMNN

SEQ ID NO: 6 - protein, 7 aa, synthetic construct
MHHHHHH

SEQ ID NO: 7 - protein, 10 aa, synthetic construct
GSAGSAAGSG

(19) United States
(12) Patent Application Publication
(10) Pub. No.: US 2026/0298912 A1
(43) Pub. Date: Oct. 1, 2026

(54) METHOD FOR MEASURING METHYLTRANSFERASE ENZYME ACTIVITY

(71) Applicant: HONDA MOTOR CO., LTD., TOKYO (JP)

(72) Inventors: Tatsuo NAKAMURA, YOKOHAMA-SHI (JP); Yuko NAKAMURA, YOKOHAMA-SHI (JP); Tadahisa NAKAMURA, WAKO-SHI (JP); Tsuyumi NISHIYAMA, WAKO-SHI (JP); Toshiaki SUMI, WAKO-SHI (JP)

(21) Appl. No.: 19/574,768

(22) Filed: Mar. 23, 2026

(30) Foreign Application Priority Data
Mar. 27, 2025 (JP) 2025-054291

(51) Int. Cl. G01N 33/53

(52) U.S. Cl. CPC G01N 33/5308; G01N 2333/91011; G01N 2333/978

(57) ABSTRACT

A novel method for measuring S-adenosylmethionine-dependent methyltransferase enzyme activity. In one aspect, this method includes: (1) a step of mixing: a protein having S-adenosylmethionine-dependent methyltransferase activity, S-adenosylmethionine, a methyl acceptor, S-adenosylhomocysteine deaminase, and (2) a step of measuring amounts of a substrate and/or a product of the S-adenosylmethionine-dependent methyltransferase activity.

### INCORPORATION BY REFERENCE

The material contained in the Sequence Listing XML file named “AIF-100-A_Sequence-Listing-revised”, created on Mar. 17, 2026, and having a file size of 8192 bytes, is hereby incorporated by reference in its entirety into the present application.

### FIELD OF THE INVENTION

The present disclosure relates to a method for measuring methyltransferase enzyme activity.

### BACKGROUND OF THE INVENTION

The quantification of S-adenosylmethionine (SAM)-dependent methyltransferase (MT) activity is widely used in basic and applied research in fields related to drug development, medical diagnosis, and biotechnology, and the improvement of this technique is an important issue. For example, changes in MT activity are deeply involved in underlying abnormal DNA methylation patterns in cancer and neurodegenerative diseases, and MT activity quantification assays play an important role in basic research and in the search for novel therapeutic targets. Accurate evaluation of MT activity is also essential in the optimization of genetic modification and process control in substance production using living organisms. Radioisotope (RI) and high-performance liquid chromatography (HPLC) methods have conventionally been used to measure MT activity and have shown very high sensitivity and reliability, but there are limitations in terms of handling of radioactive materials, instrument cost, and analysis time. In recent years, homogeneous assay technique for high-throughput screening (HTS) has been developed, enabling MT activity to be measured efficiently and rapidly. Representative techniques include AptaFluor from BellBrook Labs (Patent Literature 1), MTase-Glo by Promega (Patent Literature 2), and the HTRF EPIgeneous Methyltransferase Assay Kit from Revvity.

### CITATION LIST

### Patent Literatures

[Patent Literature 1] WO 2017/044494 A1
[Patent Literature 2] U.S. Patent Application Publication No. 2013/0109037 A1

### SUMMARY OF THE INVENTION

As described above, there are various methods for measuring the enzymatic activity of S-adenosylmethionine-dependent methyltransferase. However, there remains room for improvement in conventional methods. For example, the product of the enzymatic activity of an S-adenosylmethionine-dependent methyltransferase has a property of inhibiting the enzymatic activity itself. This property makes it difficult to accurately quantify S-adenosylmethionine-dependent methyltransferase activity by kinetic assay methods that exhibit linear enzymatic reaction progression. Therefore, an object of the present disclosure is to provide a novel method for measuring S-adenosylmethionine-dependent methyltransferase enzyme activity.

In order to achieve the above object, the present disclosure, in one aspect, includes the following inventions:

### (Invention 1)

A method for measuring methyltransferase enzyme activity,
wherein the method includes:
   (1) a step of mixing:
a protein having S-adenosylmethionine-dependent methyltransferase activity,
S-adenosylmethionine,
a methyl acceptor,
S-adenosylhomocysteine deaminase, and
   (2) a step of measuring amounts of a substrate and/or a product of the S-adenosylmethionine-dependent methyltransferase activity.

### (Invention 2)

The method of Invention 1,
wherein the step (1) further includes mixing a nucleic acid aptamer beacon, and
the step (2) includes measuring light emission of the nucleic acid aptamer beacon.

### (Invention 3)

The method of Invention 2, wherein the nucleic acid aptamer beacon is capable of detecting S-adenosylmethionine and S-adenosylhomocysteine.

In one aspect, the invention described above measures methyltransferase enzyme activity in an environment including S-adenosylhomocysteine deaminase. This can reduce the accumulation of S-adenosylhomocysteine resulting from the action of methyltransferase enzymes. S-adenosylhomocysteine has a function of inhibiting the methyltransferase enzyme activity. Thus, by reducing the accumulation of S-adenosylhomocysteine, the enzyme activity can be accurately measured.

### BRIEF DESCRIPTION OF THE DRAWINGS

FIG. 1 illustrates a methyltransferase (MT) activity measurement system using a DNA aptamer beacon according to an embodiment; Each abbreviation has the following meaning: SAM: S-adenosylmethionine; MT: methyltransferase; SAH: S-adenosylhomocysteine; SAHDA: S-adenosylhomocysteine deaminase; SIH: S-inosylhomocysteine; Interaction of the adenosine-responsive DNA aptamer beacon with the adenosine moiety in the SAM molecule results in the release of the quencher-labeled strand, which exhibits fluorescence intensity dependent on the SAM concentration; SAM is converted to SAH by methyltransferase (MT); Although the adenosine moiety in the SAH molecule can interact with the DNA aptamer beacon to enhance fluorescence, SAH is immediately degraded by SAH deaminase (SAHDA) added to the assay system to form S-inosylhomocysteine (SIH); The DNA aptamer beacon does not respond to SIH; Although SAH often causes feedback inhibition of the MT reaction even at low concentrations, the addition of SAHDA suppresses the inhibition of the reaction by SAH, allowing the MT activity to be measured more accurately; The dotted lines indicate reactions that are avoided by the addition of SAHDA;

FIG. 2 illustrates the principle of a nucleic acid aptamer beacon according to an embodiment;

FIGS. 3A-3D illustrate a response of a DNA aptamer beacon to adenosine-related compounds; Comparison of DNA aptamer beacon responses to adenosine (FIG. 3A), SAM (FIG. 3B), SAH (FIG. 3C), and inosine (FIG. 3D); Data are presented as mean±standard deviation (n=3);

FIGS. 4A-4B illustrate a calibration curve and Hanes-Woolf plot for SAM; (FIG. 4A) Calibration curve for SAM; Data are presented as mean±standard deviation (n=3); (FIG. 4B) A regression line was created using data points in a concentration range (2-20 μM) that was above the lower limit of quantification and had a coefficient of determination (R²) of 0.99 or more on a Hanes-Woolf plot, from the data used to create (FIG. 4A);

FIG. 5 illustrates SDS-PAGE analysis of SAHDA and MT proteins; Crude extracts of E. coli expressing each fusion protein and purified SAHDA and BamMHT (fused with mCherry) were separated by SDS-PAGE and analyzed by CBB staining; Arrowheads indicate the bands of purified fusion proteins;

FIGS. 6A-6B illustrate SAH degradation by SAHDA and its effect on SAM quantification; (FIG. 6A) After mixing the DNA aptamer beacon with 10 μM of SAH, the fluorescence intensity was measured with and without the addition of SAHDA, and the SAH concentration was quantified; (FIG. 6B) SAHDA was added to the SAM solution to evaluate the effect of SAHDA on the SAM calibration curve; Each data is presented as mean±standard deviation (n=3); and

FIGS. 7A-7B illustrate MT activity quantification using a DNA aptamer beacon; To measure the methyl iodide transferase activity of BamMHT, the time-dependent change in residual SAM concentration in a reaction solution containing 20 μM of SAM and 0.5 mM of KI as substrates was measured by an MT assay system using a DNA aptamer beacon; (FIG. 7A) Time-dependent change in fluorescence intensity (F−F₀) with and without BamMHT; (FIG. 7B) SAM consumption rate by BamMHT; Each data is presented as a mean value (n=3).

### DETAILED DESCRIPTION OF THE INVENTION

Specific embodiments for carrying out the present disclosure will be described in detail below. The following descriptions are intended to facilitate understanding of the invention. That is, it is not intended to limit the scope of the present disclosure.

### 1. Overview of Method

In an embodiment, the present disclosure relates to a method for measuring methyltransferase enzyme activity. The method includes the following steps:
(1) a step of mixing:
   a protein having S-adenosylmethionine-dependent methyltransferase activity,
   S-adenosylmethionine,
   a methyl acceptor,
   S-adenosylhomocysteine deaminase, and
(2) a step of measuring amounts of a substrate and/or a product of the S-adenosylmethionine-dependent methyltransferase activity.

Each step will be described below in detail.

### 2. Step of Mixing

Various components are mixed as described above. This allows specific enzymatic reactions to proceed. Specifically, an enzymatic reaction by a protein having S-adenosylmethionine-dependent methyltransferase activity and an enzymatic reaction by S-adenosylhomocysteine deaminase are allowed to proceed.

A methyl acceptor has a function of receiving a methyl group when an enzyme transfers the methyl group (or has the property of being methylated). Examples of methyl acceptors may include, but not limited to, any one or more of: nucleic acids (e.g., RNA, DNA, and the like), phospholipids (e.g., phosphatidylethanolamine), amino acids (e.g., amino acids having an amino group at a residue moiety, such as lysine and arginine), catecholamines, and halides. The halides may include, for example, one or more of iodide, bromide, and chloride. The methyl acceptor may be, for example, an organic compound or an inorganic compound. Examples of inorganic halides may include, for example, one or more of: potassium iodide, potassium bromide, potassium chloride, and the like.

Thus, additional components may be added to allow the above enzymatic reaction to proceed appropriately. For example, oxidants, reducing agents (e.g., DTT), inorganic salts (other than the methyl acceptors described above), and/or pH buffers may be added. The temperature conditions are also not particularly limited and may be adjusted to a temperature at which the enzymatic reaction proceeds appropriately. For example, the temperature for enzymatic reactions may range from 25° C. to 40° C., and preferably from 27° C. to 33° C.

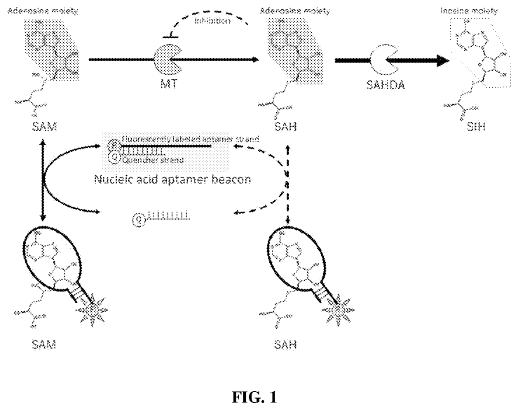

The principle of the measurement method of the present disclosure according to an embodiment is shown in FIG. 1. S-adenosylmethionine-dependent methyltransferases (MTs) (e.g., methyl halide transferases) have a function of synthesizing methyl halides from S-adenosylmethionine (SAM) and a methyl acceptor halide. In this case, the methyl group of S-adenosylmethionine is transferred. The transferring of the methyl group then results in the formation of S-adenosylhomocysteine (SAH). Here, S-adenosylhomocysteine has a function of inhibiting methyltransferase. Therefore, as the methyltransferase reaction proceeds, the reaction of methyltransferase is stopped by the reaction product S-adenosylhomocysteine. In fact, the methyltransferase reaction may be stopped at an early stage. If the reaction is stopped, it may hinder appropriate evaluation of methyltransferase performance.

Here, one of the components of the mixture includes S-adenosylhomocysteine deaminase (SAHDA). This enzyme has a function of removing the amino group of S-adenosylhomocysteine, resulting in the formation of S-inosylhomocysteine (SIH). In other words, this enzyme converts the substance having the function of inhibiting methyltransferase into other substance. The other substance, S-inosylhomocysteine, does not inhibit the methyltransferase reaction. This reduces the possibility that the methyltransferase reaction is stopped.

It should be noted that S-adenosylhomocysteine deaminase is known to occur in a wide range of species. Therefore, the species from which S-adenosylhomocysteine deaminase is derived is not particularly limited. Typically, S-adenosylhomocysteine deaminase derived from species such as Methanocaldococcus jannaschii and Thermotoga maritima may be used.

### 3. Step of Measuring

To measure the enzymatic activity of the methyltransferase described above, a change in an amount of a substrate of the enzyme and/or a change in an amount of a product of the enzyme can be measured. The measurement method is not particularly limited. For example, the measurement may be performed by the following methods: radioisotope (RI) method, high-performance liquid chromatography (HPLC) method, antibody-based method, mass spectrometry-based method, nucleic acid aptamer beacon-based method, and the like. The method using the nucleic acid aptamer beacon will be described later.

In an embodiment, a method for specifically detecting S-adenosylmethionine, a substrate of methyltransferase, may be used. A decrease in the amount of the substrate means that the enzymatic reaction is proceeding.

In another embodiment, a method for specifically detecting S-adenosylhomocysteine, a product of methyltransferase, may be used. An increase in the amount of the product means that the enzymatic reaction is proceeding.

By specifically measuring the change in either the amount of substrate or the amount of product, enzyme kinetic parameters such as Km can be determined.

It should be noted that the measurement method described above may be based on detection by identifying S-adenosylmethionine and S-adenosylhomocysteine, or it may be based on detection without identifying S-adenosylmethionine and S-adenosylhomocysteine. This is because even with the method based on the latter, S-adenosylhomocysteine is eventually converted to the other substance by S-adenosylhomocysteine deaminase.

Therefore, even if the measurement method described above is based on detection without identifying S-adenosylmethionine and S-adenosylhomocysteine, it does not interfere with measuring the enzymatic activity of methyltransferase.

However, if the measurement method described above detects S-inosylhomocysteine, it may interfere with measuring the enzymatic activity of methyltransferase. Therefore, in a preferable embodiment, the measurement method used in the measurement step satisfies the following conditions (1) and (2):
(Condition 1) at least S-adenosylmethionine is detected; and
(Condition 2) S-inosylhomocysteine is not detected.

### 4. Nucleic Acid Aptamer Beacon

In an embodiment, the measurement method described above may be based on a nucleic acid aptamer beacon. For example, in the method for measuring methyltransferase enzyme activity, the step (1) of mixing may further include mixing a nucleic acid aptamer beacon, and step (2) of measuring may include measuring light emission of the nucleic acid aptamer beacon.

An overview of the nucleic acid aptamer beacon will be described below.

Functionally, the nucleic acid aptamer beacon has a function of causing a conformational change in the nucleic acid when it binds to the target substance and presenting the change in a form that is easily recognized.

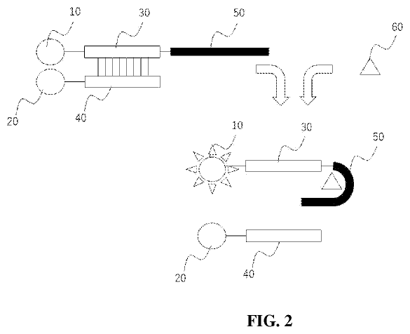

In one embodiment, the nucleic acid aptamer beacon has the following moieties (see FIG. 2):
First substance (10)
Second substance (20)
Region (30) of the first nucleic acid
Region (40) of the second nucleic acid
Target substance-binding region (50)

The first substance (10) has a function of emitting fluorescence. Here, the emitting of fluorescence refers to the absorption of light at one wavelength and the emission of light at another wavelength. The first substance (10) may also function as a donor molecule in terms of a phenomenon called FRET (Fluorescence Resonance Energy Transfer).

The second substance (20) has a function of modulating the light emission by the first substance (10). For example, the second substance (20) may function as a quencher (also referred to herein as a quenching agent). Specifically, the second substance (20) may absorb light emission at a specific wavelength by the first substance (10) and may not emit light at that wavelength or another wavelength. This allows the light emission by the first substance (10) to be quenched by the second substance (20).

The second substance (20) may also function as an acceptor molecule in terms of a phenomenon called FRET (Fluorescence Resonance Energy Transfer). Specifically, the second substance (20) can absorb light emission at a specific wavelength by the first substance (10) and emit light having another wavelength.

The region of the first nucleic acid (30) is a region of nucleic acid linked directly or indirectly to the first substance (10). The region of the second nucleic acid (40) is a region of nucleic acid linked directly or indirectly to the second substance (20).

In one example, “directly linked” may mean, for example, that the first substance (10), which is a substance other than a nucleic acid, is linked to the region of the first nucleic acid (30), which is a nucleic acid, without intervening another atom or molecule. The same is true for the second substance (20).

In another example, “indirectly linked” may mean, for example, that the first substance (10), which is a substance other than a nucleic acid, is linked to the region (30) of the first nucleic acid, which is a nucleic acid, via another substance (e.g., another atom, another molecule, such as another nucleic acid, or another linker molecule). The same is true for the second substance (20).

The target substance-binding region (50) is a nucleic acid region that binds to the target substance (60). Preferably, it is a nucleic acid region that specifically binds to the target substance (60). By way of non-limiting example, “specifically bind” may mean that the nature of binding (e.g., binding constant, and the like) to the target substance (60) differs to the extent that it produces a statistically significant difference as compared to a control.

With reference to FIG. 2, how each of the above-mentioned elements contributes to the detection of the target substance (60) will be described.

In one example, the region (30) of the first nucleic acid and the region (40) of the second nucleic acid are hybridized. A distance between the first substance (10) and the second substance (20), each linked to these regions, decreases. Thus, the light emission from the first substance (10) is modulated by the second substance (20). For example, when the second substance (20) functions as a quencher, the light emission from the first substance (10) is quenched.

However, if a target substance (60) is present, such a target substance (60) binds to the target substance-binding region (50). This causes at least a partial conformational change in the nucleic acid aptamer beacon. When the conformational change occurs, the region (30) of the first nucleic acid and the region (40) of the second nucleic acid are prevented from hybridizing. This results in an increased distance between the first substance (10) and the second substance (20). Thus, the light emission from the first substance (10) is not modulated by the second substance (20).

The above descriptions are only illustrative, and various modification patterns from the structure shown in FIG. 2 are present.

### 4-1. Types of Nucleic Acids (Modification Pattern 1)

The nucleic acid used may be DNA, RNA, or a combination of both (e.g., the region (30) of the first nucleic acid is DNA and the region (40) of the second nucleic acid is RNA, or the region (30) of the first nucleic acid is RNA and the region (40) of the second nucleic acid is DNA).

### 4-2. Function of First and Second Substances (Modification Pattern 2)

In one example, the first substance (10) and the second substance (20) may be a combination in which donor fluorescence is quenched via FRET. Here, quenching also includes events where fluorescence is partially attenuated. For example, the first substance (10) has a function of emitting light at a certain wavelength. The second substance (20) then has a function of absorbing light at or near that wavelength (and the second substance (20) does not have a function of emitting light).

In another example, the first substance (10) and the second substance (20) may be a combination that induces fluorescence emission at an acceptor via FRET (Fluorescence Resonance Energy Transfer). Here, the first substance (10) may function as a donor molecule and the second substance (20) may function as an acceptor molecule. For example, the first substance (10) has a function of emitting light at a certain wavelength. The second substance (20) then has a function of absorbing light at or near that wavelength. The second substance (20) can also use light at or near that wavelength as excitation light and has a function of emitting light at a different wavelength.

As another example, the first substance (10) and the second substance (20) may serve as a donor and an acceptor, respectively, in PET (Photoinduced Electron Transfer).

### 4-3. Region of First Nucleic Acid and Region of Second Nucleic Acid (Modification Pattern 3)

The region (30) of the first nucleic acid and the region (40) of the second nucleic acid may be integrated or may be present separately. FIG. 2 is an example of the latter. When they are integrated, the region (30) of the first nucleic acid and the region (40) of the second nucleic acid may be present, for example, so as to sandwich the target substance-binding region (50) and/or another nucleic acid region. When both are at least partially hybridized, the nucleic acid aptamer beacon may have a loop structure.

### 4-4. Distance Between First Substance and Second Substance (Modification Pattern 4)

In the example in FIG. 2, when the target substance (60) is not present, the distance between the first substance (10) and the second substance (20) decreases, and when the target substance (60) is present, the distance between the first substance (10) and the second substance (20) increases. In another example, when the target substance (60) is not present, the distance between the first substance (10) and the second substance (20) may increase, and when the target substance (60) is present, the distance between the first substance (10) and the second substance (20) may decrease.

As described above, nucleic acid aptamer beacons can be modified according to various modification patterns or combinations thereof.

### 4-5. Combination of First and Second Substances

The combination of the first substance and the second substance is not particularly limited, and may be, for example, a combination capable of causing FRET. In view of homo-FRET, the combination of the first and second substances may include not only combinations of different types of substances but also combinations of the same type of substance. Typically, however, the combination of the first and second substances is a combination of different types of substances. The second substance may also include a quenching agent.

By way of non-limiting example, the following combinations are included:
CFP and YFP
GFP (including EGFP) and mCherry
mTurquoise2 and m Venus
mNeonGreen and mRuby3
Fluorescein and Tetramethylrhodamine
Fluorescein and Black Hole Quencher® 1 (BHQ-1)
IAEDANS (5-[2-Iodoacetylaminoethyl] Aminonaphthalene-1-Sulfonic Acid) and Fluorescein
EDANS (5-(2-Aminoethylamino) naphthalene-1-sulfonic acid) and Dabcyl Cy3 and Cy5
Alexa Fluor 488 and Alexa Fluor 594.

In another example, the combination may be capable of causing PET.

By way of non-limiting example, the following combinations are included:
Porphyrins and quinones
Organic dyes and amines

The term “nucleic acid” as used herein may or may not include naturally occurring nucleic acids. Additionally or alternatively, the term “nucleic acid” as used herein may or may not include chemically modified nucleic acids. The same is true for the terms “DNA” and “RNA” as used herein. The type of chemical modification is not particularly limited, but it may include, for example, one or more of: fluorescent labeling (e.g., FAM, TAMRA, ROX, and the like); methylation (e.g., 5-methylcytosine, methylation of the 2′-OH group of RNA, and the like); introduction of base analogs (e.g., 5-bromouracil (BrU), 6-thioguanine (6-TG), pseudouridine, and the like); fluoro-modification (e.g., 2′-fluoro-RNA (2′-F RNA)); introduction of linkers (e.g., PEGylation, biotin conjugation, and the like); polyadenylation (Poly (A) tail); morpholino nucleic acid (PMO); peptide nucleic acid (PNA); locked nucleotide (LNA); bridged nucleotide (BNA); unlocked nucleotide (UNA); glycol nucleotide (GNA); phosphorothioate bond; alkyl phosphonate diester modification; and the like.

### 4-3. Step of Measuring

As described above, the step (2) of measuring may include measuring light emission of the nucleic acid aptamer beacon. The step (2) of measuring may include, for example, irradiating the mixture with light at or near the excitation wavelength of the first substance. More specifically, the step (2) may measure the light emission from the first substance or the second substance using a microplate reader or the like, or may measure the effect of quenching by the second substance.

### 4-7. Nucleic Acid Aptamer Beacon in Method for Measuring Enzymatic Activity of Methyltransferase

With reference back to FIG. 1, how the nucleic acid aptamer beacon contributes to the measurement will be described. As described above, S-adenosylmethionine and S-adenosylhomocysteine are present as the substrate and the product of methyltransferase, respectively. The fluorescently labeled aptamer strand making up the nucleic acid aptamer beacon can bind to these substances. On the other hand, the fluorescently labeled aptamer strand cannot bind to S-inosylhomocysteine. In other words, the fluorescently labeled aptamer strand has a function of specifically binding to an adenosine moiety.

When S-adenosylmethionine and S-adenosylhomocysteine are not present, the fluorescently labeled aptamer strand hybridizes to the quencher strand. As a result, the first substance, the fluorescent moiety (labeled “F” in FIG. 2), and the second substance, the quencher moiety (labeled “Q” in FIG. 2), come closer to each other. As a result, the light emission from the fluorescent moiety is quenched.

When one or more of S-adenosylmethionine and S-adenosylhomocysteine are present, the fluorescently labeled aptamer strand undergoes a conformational change, resulting in the release of the quencher strand. As a result of the absence of the quencher moiety in close proximity, the light emission from the fluorescent moiety can be detected.

Under normal circumstances, it is not possible to measure enzyme activity based solely on the nucleic acid aptamer beacon because the fluorescently labeled aptamer strand binds specifically to both the substrate and the product of methyltransferase. However, S-adenosylhomocysteine is converted to S-inosylhomocysteine in the presence of S-adenosylhomocysteine deaminase. Thus, consequently, the fluorescently labeled aptamer strand detects only the substrate of methyltransferase. This enables the measurement of enzyme activity.

By way of non-limiting example, the nucleic acid aptamer beacon may include the following nucleic acid sequences as target substance-binding regions. The following sequences correspond to regions that specifically recognize an adenosine moiety.

(SEQ ID NO: 1)
5′-ACGACGTTTGCGATGAGAAACGTATGGTTTCGAAGGTCGT-3′

### EXAMPLES

### 1. Preparation of DNA Aptamer Beacon

In this example, a DNA aptamer beacon (Ding and Liu, 2023) that specifically detects adenosine was used as a model. The DNA aptamer beacon was comprised of a fluorescein (FAM)-labeled aptamer strand and a quencher (Black Hole Quencher1, BHQ1)-labeled strand (Table 1), each synthesized by Eurofins Genomics Co., Ltd. 1 μM of FAM-labeled aptamer strand, 2 μM of quencher-labeled strand, 50 mM of Tris-HCl (pH 7.5), 500 mM of NaCl, and 20 mM of MgCl₂ were mixed and heated at 95° C. for 2 minutes, and then the temperature was lowered to 25° C. over 30 minutes to hybridize both DNA strands, thereby preparing a DNA aptamer beacon solution.

TABLE 1
Nucleotide Sequences and Modification Sites of Oligo DNAs Used

| Oligo DNA Name | Nucleotide Sequence and Modification Sites |
|---|---|
| FAM-labeled aptamer | 5′- |
| strand | [FAM]CTCTCGACGACGTTTGCGATGAGAAACGTATGGTTTCG |
| | AAGGTCGTC-3 (SEQ ID NO: 2) |
| Quencher-labeled | 5′-AGTCGTCGAGAG[BHQ1]-3′ (SEQ ID NO: 3) |
| strand | |

### 2. Analysis of Response of DNA Aptamer Beacons to Adenosine-Related Compounds

Detection of adenosine-related compounds using a DNA aptamer beacon was performed in a polystyrene 96-well plate format, and fluorescence measurements (Ex 480 nm, Em 530 nm) were made at 30° C. using a plate reader (Synergy H1, BioTek). The volume of the liquid in each well was 100 μL, and the assay solution contained adenosine-related compounds (at each concentration), DNA aptamer beacon (20 nM as FAM-labeled aptamer strand), and 100 mM Tris-acetate (pH 7.5). Adenosine, S-adenosylmethionine (SAM), S-adenosylhomocysteine (SAH), and inosine were used as adenosine-related compounds.

### 3. Preparation of DNA Constructs and Transformation of E. coli

A plasmid containing a DNA fragment encoding SAH deaminase (SAHDA, TM0936) (Hermann et al., 2007) from Thermotoga maritima was obtained from the DNASU plasmid repository (Table 2). To add 7 amino acid residues including a 6×His tag (Met-His-His-His-His-His-His) to the N-terminus of SAHDA and express it in E. coli, the DNA fragment encoding SAHDA was amplified by PCR using the above-described plasmid as a template, cloned downstream of the T7 promoter of the pDEST17 vector, and introduced into E. coli BL21 (DE3) strain.

Methyl Halide Transferase (BamMHT) (Ni and Hager, 1998) from Batis maritima was used as a model for MT activity quantification (Table 2). The DNA fragment encoding BamMHT was synthesized by Integrated DNA Technologies, Inc. The DNA fragment was cloned downstream of the T7 promoter of the pDEST17 vector for expression in E. coli, with 7 amino acid residues including 6×His tag (Met-His-His-His-His-His-His) added to the N-terminus and a fluorescent protein (mCherry) fused to the C-terminus via a 10 amino acid residue linker (Gly-Ser-Ala-Gly-Ser-Ala-Ala-Gly-Ser-Gly), and was introduced into E. coli BL21 (DE3) strain.

TABLE 2
Amino Acid Sequences of Proteins Used
SAHDA (from Thermotoga maritima, Theoretical pI/Mw: 5.15/45772.51)
MIIGNCLILKDFSSEPFWGAVEIENGTIKRVLQGEVKVDLDLSGKLVMPA
LFNTHTHAPMTLLRGVAEDLSFEEWLFSKVLPIEDRLTEKMAYYGTILA
QMEMARHGIAGFVDMYFHEEWIAKAVRDFGMRALLTRGLVDSNGDDG
GRLEENLKLYNEWNGFEGRIFVGFGPHSPYLCSEEYLKRVEDTAKSLNA
PVTIHLYETSKEEYDLEDILNIGLKEVKTIAAHCVHLPERYFGVLKDIPFF
VSHNPASNLKLGNGIAPVQRMIEHGMKVTLGTDGAASNNSLNLFFEMR
LASLLQKAQNPRNLDVNTCLKMVTYDGAQAMGFKSGKIEEGWNADLV
VIDLDLPEMFPVQNIKNHLVHAFSGEVFATMVAGKWIYFDGEYPTIDSE
EVKRELARIEKELYSS (SEQ ID NO: 4)
BamMHT (from Batis maritima, Theoretical pI/Mw: 5.12/25786.78)
MSTVANIAPVFTGDCKTIPTPEECATFLYKVVNSGGWEKCWVEEVIPWD
LGVPTPLVLHLVKNNALPNGKGLVPGCGGGYDVVAMANPERFMVGLD
ISENALKKARETFSTMPNSSCFSFVKEDVFTWRPEQPFDFIFDYVFFCAID
PKMRPAWGKAMYELLKPDGELITLMYPITNHEGGPPFSVSESEYEKVLV
PLGFKQLSLEDYSDLAVEPRKGKEKLARWKKMNN (SEQ ID NO: 5)

### 4. Expression and Purification of Fusion Proteins in E. coli

Each fusion protein-expressing E. coli was cultured overnight at 30° C. in ZYM5052 medium (Studier, 2005) containing 100 μg/ml of ampicillin, and the E. coli cells were collected. The collected bacterial cells were suspended in 15 ml of binding buffer according to the protocol of the His-Bind Kit (Novagen), and the cells were disrupted using a pressure cell disruption machine (EmulsiFlex-B15, AVESTIN). After centrifugation (16,000 g, 4° C., 10 minutes), the supernatant was regarded as a crude protein extract. The target protein was purified from the crude protein extract by a batch method using the His-Bind Kit resin. After desalting and concentration by ammonium sulfate precipitation and ultrafiltration column (10 KD, GVS), SAHDA and BamMHT solutions were prepared to contain 0.1 M of Tris-acetate (pH 7.5), 1 mM of DTT, and 50% of glycerol. Purified proteins were separated by 10% sodium dodecyl sulfate-polyacrylamide gel electrophoresis (SDS-PAGE) and confirmed by staining them with Coomassie Brilliant Blue (CBB). Purified protein concentrations were determined by the Bradford method using bovine serum albumin (BSA) as the standard protein.

### 5. MT Activity Measurement System Using DNA Aptamer Beacon

Quantification of MT activity using a DNA aptamer beacon was performed in a polystyrene 96-well plate format, and fluorescence measurements (Ex 480 nm, Em 530 nm) were made at 30° C. using a plate reader (Synergy H1, BioTek). The volume of the liquid in each well was 100 μL, and 20 μM of SAM, DNA aptamer beacon (20 nM as FAM-labeled aptamer strand), 100 mM of Tris-acetate (pH 7.5), 0.5 mM of KI, 1 mM of dithiothreitol (DTT), 4 μg of purified SAHDA, and 1 μg of purified BamMHT were mixed. The enzymatic reaction was initiated by the addition of KI, and MT activity was calculated based on data in the concentration range where the rate of decrease of SAM concentration was constant, using the kinetic assay method.

### 6. Results

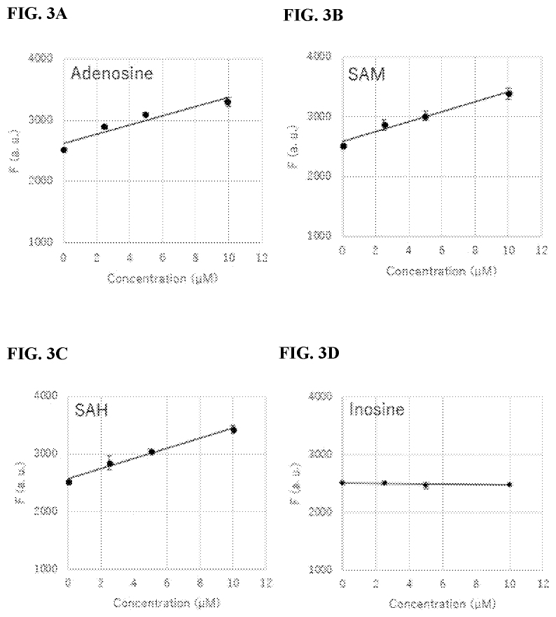

A DNA aptamer beacon (Ade1301b) (Ding and Liu, 2023) for quantifying adenosine has previously been reported. To examine the applicability of this DNA aptamer beacon to the assay system shown in FIG. 1, whether it responded to related compounds other than adenosine was analyzed (FIG. 3). The results showed that the DNA aptamer beacon also responded to S-adenosylmethionine and S-adenosylhomocysteine, which had an intramolecular adenosine structure, at levels comparable to those for adenosine. It was also shown that the beacon did not respond to inosine, which was structurally similar to adenosine but did not have an amino group.

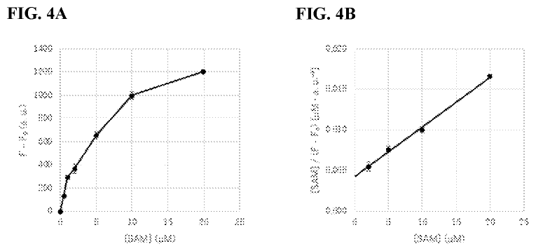

A calibration curve for SAM using this DNA aptamer beacon and a Hanes-Woolf plot based thereon were created (FIGS. 4A-4B). The limit of detection (3.3σ/S) and the limit of quantification (10σ/S) were calculated from the standard deviation (n=3) of the fluorescence intensity (F₀) of the blank value (0 μM of SAM) and the slope (S) of the linear concentration range (0-2 μM) in the calibration curve (Table 3). Apparent dissociation constants were calculated from regression lines based on Hanes-Woolf plots (FIG. 4B) created from the SAM calibration curve data (FIG. 4A) (Table 3).

TABLE 3
Characteristics of SAM Quantification
System Using DNA Aptamer Beacon

| | |
|---|---|
| Detection Limit (μM) | 0.61 |
| Limit of Quantification (μM) | 1.85 |
| Apparent Dissociation Constant (μM) | 7.09 |

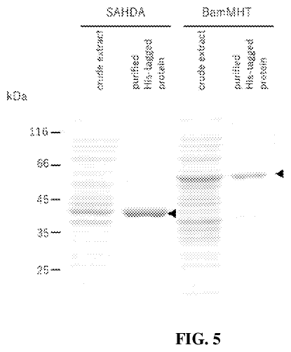

The gene for the SAH-degrading enzyme (SAH deaminase) used to eliminate SAH produced by MT activity from the reaction system was expressed in E. coli and the fusion protein was purified (FIG. 5). The fusion BamMHT protein used as a model for the MT assay system was similarly expressed in E. coli and purified (FIG. 5). Both of the proteins exhibited the expected molecular weight and were prepared with sufficient purity.

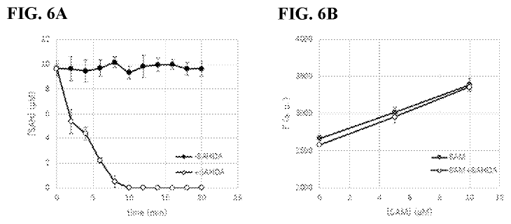

It was analyzed whether the prepared fusion SAHDA protein sufficiently degrades SAH in the reaction system. The results showed that the quantitative value of SAH in the system did not change for the negative control that did not contain SAHDA, whereas SAH was degraded and disappeared for the sample that contained SAHDA (FIG. 6A). The addition of SAHDA did not affect SAM quantification using the DNA aptamer beacon (FIG. 6B).

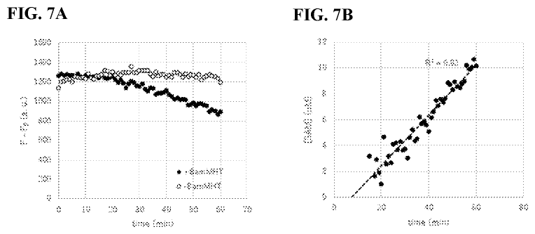

To confirm whether it was possible to construct the MT activity quantification system shown in FIG. 1, the methyl group transfer activity of the BamMHT protein to iodide ions was measured as a model for MT proteins. No decrease in fluorescence intensity over time was observed for the negative control that did not contain BamMHT protein, whereas a decrease in fluorescence value (F−F₀) due to SAM consumption was observed for the sample that contained BamMHT protein (FIG. 7A). When an amount of SAM consumption was calculated from these fluorescence values using the SAM calibration curve, a linear increase in the amount of SAM consumption was observed (FIG. 7B). Even in a reaction system showing a decrease in SAM concentration of at least 10 μM, the enzymatic reaction was shown to proceed linearly; this is a concentration range in which MT activity would be greatly inhibited by SAH that would otherwise be accumulated if there is no SAHDA. The absence of the decrease in fluorescence immediately after the start of the enzymatic reaction would be because a certain amount of time would be required for the equilibrium of the interaction between SAM and the DNA aptamer to be reached.

The present disclosure provides a novel approach to overcome various challenges of conventional techniques, such as the difficulty of accurately calculating enzyme activity due to the reliance on endpoint measurements, reproducibility problems caused by the stability of RNA and antibodies, and operational complexity and reproducibility problems due to multi-step enzyme reactions. Particularly, in the present disclosure, it is discovered for the first time that the DNA aptamer beacon (Ade1301b) (Ding and Liu, 2023) also responds to SAM and SAH. Furthermore, in the reaction system, SAH is rapidly degraded by a single enzyme (SAHDA), and the product SIH does not affect the DNA aptamer beacon. Removal of SAH reduces inhibition of MT activity, enabling accurate quantification of MT activity by a kinetic assay method that shows linear enzymatic reaction progression.

In the above Examples, the following literature was consulted.

### REFERENCES

### Non-Patent Literature

Ding Y, Liu J (2023) Pushing adenosine and ATP SELEX for DNA Aptamers with nanomolar affinity. J Am Chem Soc 145:7540-7547
Hermann J C, Marti-Arbona R, Fedorov A A, Fedorov E, Almo S C, Shoichet B K, Raushel F M (2007) Structure-based activity prediction for an enzyme of unknown function. Nature 448:775-779
Ni X, Hager L P (1998) cDNA cloning of Batis maritima methyl chloride transferase and purification of the enzyme. Proc Natl Acad Sci 95:12866-12871 Studier FW (2005) Protein production by auto-induction in high-density shaking cultures. Protein Expr Purif 41:207-234

The specific embodiments according to the present disclosure have been described above. The above embodiments are only specific examples, and the present disclosure is not limited to the above embodiments. For example, the technical features disclosed in one of the above embodiments can be applied to other embodiments. Unless otherwise noted, for a particular method, some steps may be interchanged with the order of other steps, and further steps may be added between two specific steps. The scope of this invention is defined by the claims.

### DESCRIPTION OF REFERENCE NUMERALS

10 region of first substance
20 region of second substance
30 region of first nucleic acid
40 region of second nucleic acid
50 target substance-binding region
60 target substance

1. A method for measuring methyltransferase enzyme activity,
wherein the method comprises:
(1) a step of mixing:
a protein having S-adenosylmethionine-dependent methyltransferase activity,
S-adenosylmethionine,
a methyl acceptor,
S-adenosylhomocysteine deaminase, and
(2) a step of measuring amounts of a substrate and/or a product of the S-adenosylmethionine-dependent methyltransferase activity.

2. The method of claim 1,
wherein the step (1) further comprises mixing a nucleic acid aptamer beacon, and
the step (2) comprises measuring light emission of the nucleic acid aptamer beacon.

3. The method of claim 2, wherein the nucleic acid aptamer beacon is capable of detecting S-adenosylmethionine and S-adenosylhomocysteine.
